(19) United States
(12) Patent Application Publication
(10) Pub. No.: US 2004/0265353 A1
(43) Pub. Date: Dec. 30, 2004

(54) Systems and methods for treating panic attacks

(75) Inventors: Jie Zhang, Salt Lake City, UT (US); Kevin S. Warner, West Jordan, UT (US); Michael A. Ashburn, Salt Lake City, UT (US)

Correspondence Address: THORPE NORTH & WESTERN, LLP., 8180 SOUTH 700 EAST, SUITE 200, P.O. BOX 1219, SANDY, UT, 84070, US

(73) Assignee: Zars, Inc.

(21) Appl. No.: 10/856,749

(22) Filed: May 28, 2004

Related U.S. Application Data
(63) Continuation-in-part of application No. 09/878,558, filed on Jun. 11, 2001, now Pat. No. 6,756,053.
Application No. 09/878,558 is a continuation-in-part of application No. 09/162,890, filed on Sep. 29, 1998, now Pat. No. 6,245,347.
Application No. 09/162,890 is a continuation-in-part of application No. 08/819,880, filed on Mar. 18, 1997, now Pat. No. 5,919,479.
Application No. 08/819,880 is a division of application No. 08/508,463, filed on Jul. 28, 1995, now Pat. No. 5,658,583.

(51) Int. Cl. A61K 31/66; A61K 9/70; A61N 1/30

(52) U.S. Cl. 424/423; 424/449; 604/20

(57) ABSTRACT

Systems and methods for attenuating a panic attack are provided. Such methods can comprise steps of delivering a drug to a subject such that the drug forms a depot beneath a skin surface, wherein the drug is suitable for attenuating a panic attack if delivered in a pharmaceutically effective amount into systemic circulation of the subject; and applying heat to the skin surface when the subject experiences an onset of the panic attack, thereby causing the pharmaceutically effective amount of the drug to rapidly enter systemic circulation from the depot.

The present application is a continuation-in-part application of U.S. patent application Ser. No. 09/878,558, filed on Jun. 11, 2001, which is a continuation-in-part application of U.S. application Ser. No. 09/162,890, filed Sep. 29, 1998, which is a continuation in part of U.S. application Ser. No. 08/819,880, filed Mar. 18, 1997, now U.S. Pat. No. 5,919,479, which is divisional application of U.S. application Ser. No. 08/508,463, filed Jul. 28, 1995, now U.S. Pat. No. 5,658,583, each of which are incorporated herein by reference in its entirety.

### FIELD OF THE INVENTION

The present invention is drawn to systems and methods for attenuating or aborting panic attacks at their onset. The present invention is also related to systems and methods that provide prevention of panic attacks through the delivery of baseline levels of drug, as well as attenuation of ongoing panic attacks through rapid delivery of an increased dose of drug into a patient's systemic circulation at or shortly after the onset of a panic attack.

### BACKGROUND OF THE INVENTION

Millions of people suffer from panic attacks (panic disorder). Some people who suffer from panic disorder experience symptoms such as heart attack or sensations of insanity. Other symptoms can include heart palpitations, chest pain or discomfort, sweating, trembling, tingling sensations, feeling of choking, fear of dying, fear of losing control, and feelings out of step with reality. Panic disorder often occurs with agoraphobia, which are fears of open and/or closed spaces, crowded places, unfamiliar places, and being alone, often causing a loss of a sense of security.

A typical panic attack has dramatic and acute symptoms which typically peak within about 10 minutes, but may last for hours. Symptoms can be perceived by the patient as medical symptoms, and are often characteristic of strong autonomic discharge, e.g., heart pounding, chest pain, trembling, choking, abdominal pain, sweating, dizziness, disorganization, confusion, dread, sense of impending doom or terror, etc. Attacks may be initiated by a triggering event, such as crowds, closed spaces, etc. Panic attacks may be rare or can occur several times a day.

Current therapies focus on using drugs to prevent the occurrence of panic attacks rather than to attenuate or abort on-going attacks. Those therapies include the use of drugs of various types, including benzodiazepines, e.g., lorazepam, prazepam, flurazepam, clonazepam, triazolam, chlordiazepoxide, halazepam, temazepam, oxazepam, clorazepate, diazepam, and alprazolam; monoamine oxidase inhibitors (MAOs), such as selegilene, isocarboxid, phenelzine and tranylcypromine; selective serotonin reuptake inhibitors (SSRIs) such as paroxetine, fluoxetine, and sertraline; tricyclic antidepressants such as clomiprimine, nortriptyline, amitriptyline, imipramine, and desipramine; and other antidepressants such as venlafaxine and nefaxadone. Though effective in reducing panic attack occurrence, none of these drugs have been shown effective in completely and safely preventing panic attacks. One approach that has been tried has been to use higher doses of some of the previously mentioned drugs. Though the delivery of a higher dose of a drug can be effective in preventing panic attacks, doses of drugs are usually limited by their adverse side effects. For example, too high of a dose of a benzodiazepine can cause severe drowsiness and complications related to the cardiovascular system. Therefore, more effective prevention through higher ongoing dosing is not usually practical.

As there are no drugs currently available that can completely prevent panic attacks, and as panic attacks are very disruptive to the lives of patients, it would be desirable to provide methods or drugs that can be administered to provide relief to patients who suffer from panic attacks as they are occurring.

### SUMMARY OF THE INVENTION

It has been recognized that systems and methods for attenuating or aborting attacks at their onset would be beneficial to patients suffering from infrequent to frequent panic attacks. It would also be beneficial to reduce the occurrences of panic attacks.

In accordance with these recognitions, a method for attenuating a panic attack can comprise steps of delivering a drug to a subject such that the drug forms a depot beneath a skin surface, wherein the drug is suitable for attenuating a panic attack if delivered in a pharmaceutically effective amount into systemic circulation of the subject. Once the depot is formed, the step of applying heat or controlled heat to the skin surface when the subject experiences an onset of the panic attack can be carried out, thereby causing the pharmaceutically effective dose of the drug to enter systemic circulation from the depot.

In another embodiment, a method for reducing the occurrence of panic attacks as well as attenuate panic attacks at their onset can comprise steps of delivering a drug to a subject having a history of panic attacks such that the drug generates a baseline level of the drug within systemic circulation of the subject, and forms a depot of the drug beneath a skin surface of the subject that is deliverable into systemic circulation upon application of heat. The drug can be suitable for reducing the occurrences of panic attacks when delivered at the baseline level, and can also be suitable for attenuating the symptoms of a panic attack upon onset when an additional dose is delivered from the depot into systemic circulation. The additional dose from the depot can cause a rapid increase in the drug's concentration in systemic circulation. A step of applying heat, such as in the form of controlled heat, to the skin surface when the symptoms of the panic attack are experienced by the subject can be carried out to treat the onset of the panic attack.

In another embodiment, a system for attenuating a panic attack can comprise a drug delivery device and a heating device. The drug delivery device can be configured to form a depot of drug beneath a skin surface of the subject. The drug can be suitable for attenuating a panic attack if delivered in a pharmaceutically effective amount into systemic circulation of the subject. The heating device can be configured to apply heat to the skin surface upon the onset of the panic attack, thereby causing the pharmaceutically effective amount of the drug to enter systemic circulation from the depot.

In still another embodiment, a system for reducing the occurrence of panic attacks as well as attenuate panic attacks at their onset can comprise a drug delivery device and a heating device. The drug delivery device can be configured to generate a baseline level of the drug within systemic circulation of a subject, and form a depot of the drug beneath a skin surface of the subject that is deliverable into systemic circulation upon demand to rapidly cause an increased drug level in the patient upon application of heat. The drug can be suitable for reducing the occurrences of panic attacks when delivered at the baseline level, and can also be suitable for attenuating the symptoms of a panic attack upon onset when an extra dose of the drug is delivered from the depot into systemic circulation. The heating device can be configured to apply heat, such as in the form of controlled heat, to the skin surface upon the onset of the panic attack.

Additional features and advantages of the invention will be apparent from the following detailed description which illustrates, by way of example, features of the invention.

### BRIED DESCRIPTION OF THE DRAWINGS

FIG. 1 is a schematic representation of a reservoir patch adhered to a skin surface, wherein the reservoir includes a non-gelled microemulsion including a benzodiazepine;

FIG. 2 is a schematic representation of a matrix patch adhered to a skin surface, wherein the matrix includes a benzodiazepine and an adhesive matrix;

FIG. 3 is a schematic representation of a gel patch adhered to a skin surface, wherein the gel includes a benzodiazepine;

FIG. 4 is a schematic representation of a Controlled Heat-Assisted Drug Delivery (hereinafter “CHADD”) heating device that can be used in accordance with embodiments of the present invention; and

FIG. 5 is an average temperature profile from three independent applications of a CHADD heating patch as tested on human skin.

### DETAILED DESCRIPTION OF THE PREFERRED EMBODIMENT(S)

Before the present invention is disclosed and described, it is to be understood that this invention is not limited to the particular process steps and materials disclosed herein because such process steps and materials may vary somewhat. It is also to be understood that the terminology used herein is used for the purpose of describing particular embodiments only. The terms are not intended to be limiting because the scope of the present invention is intended to be limited only by the appended claims and equivalents thereof.

As used in this specification and the appended claims, the singular forms “a,” “an,” and “the” include plural referents unless the content clearly dictates otherwise.

The term “baseline” or “baseline level” refers to the delivery of relatively constant concentrations of the drug into systemic circulation of a patient. The baseline levels of a particular drug can depend on the drug, the delivery system, and/or the individual patient.

The term “depot” refers to the collection of a drug beneath a skin surface. The depot can be formed by injecting or implanting, or by transdermal delivery. In transdermal delivery, the drug permeates across the outer barrier of the skin, i.e. the stratum corneum, to reach the viable epidermis and dermis layers. For many drugs, a portion of the drug then gets absorbed by the capillary blood vessels and enters the systemic circulation while another portion gets stored in tissues close to the skin surface to form a depot. After sufficient application time, the transdermal delivery system can reach a relatively steady state transport of the drug, meaning the rate at which the drug enters the depot will approximately equal the rate at which the drug leaves the depot. At that point, the depot is said to be fully populated or fully formed. The depot acts as a reservoir of drug that both receives and passes the drug into systemic circulation. When heat is applied to the skin surface directly above the depot, blood circulation through and around the depot is increased dramatically. Thus, almost immediately, the drug from the depot is delivered into the systemic circulation. Since this bolus of drug does not involve a transdermal permeation process, the extra dose of the drug is delivered into the systemic circulation at a speed much faster than occurs with typical transdermal processes, and close to as fast as that which occurs with intravenous injection. The magnitude of the depot can depend on the delivery rate and the nature of the drug molecule, such as its lipophilicity. For a given drug, the transdermal delivery system is configured to deliver a sufficient amount of the drug to form a useful depot. By this technique, a pharmaceutically effective amount of the drug to attenuate a panic attack can be rapidly delivered into systemic circulation.

The term “reservoir patch” refers to a transdermal delivery system that typically includes four layers. The four layers include an impermeable backing film which gives mechanical support; a liquid compartment containing a drug solution, gel, or suspension; a semi permeable membrane; and an adhesive layer that contacts and adheres to the skin surface.

In contrast, a “single-layer drug-in-adhesive patch,” which is a type of matrix patch, includes the drug directly within a skin-contacting adhesive. The adhesive in this formulation can serve two functions: first, to affix the system to the skin, and second, to serve as a foundation containing drug and any other ingredients or excipients under a backing film.

Another type of patch is a “semisolid patch” or “gel patch.” This type of patch includes a semisolid phase or hydrogel that contains a drug suspension. The drug-containing semisolid phase or hydrogel is typically in direct contact with the skin. A skin adhesion component can either be incorporated into the drug suspension or hydrogel itself, or can be present in a concentric or perimeter configuration around the drug-containing semisolid phase or hydrogel. In one embodiment, gel patch can carry a gelled emulsion, and in another embodiment, the gel patch can carry a gelled microemulsion.

The terms “controlled heating” and “controlled heat” are defined as heat application that is capable of heating a skin surface to pre-determined narrow temperature range for a predetermined duration.

The term “microemulsion” can be defined as a system of water, oil, and surfactant(s), which when mixed in certain weight ratios, typically form a clear or otherwise transparent and thermodynamically stable liquid. Typically, a microemulsion is transparent because the oil droplets are smaller than the wavelengths of visible light, e.g., approximately from 400 nm to 800 nm. In one embodiment, the microemulsion can be gelled and included in a drug delivery matrix patch, reservoir patch, or gel patch. If not gelled, the microemulsion can be included in a reservoir patch.

The term “emulsion” can be defined as a system including a continuous phase and a discontinuous phase. Typically, dispersed droplets (discontinuous phase) can be present in another liquid (continuous phase). An emulsifying agent may or may not also be present. The consistency of an emulsified system may range form a relatively low viscosity system, e.g., lotions, to more semisolid systems, e.g., creams. These emulsions can be included in a drug delivery matrix patch, reservoir patch, or gel patch.

While some known drugs may be effective to attenuate or even abort the onset of panic attacks, due to unpredictable nature and the short duration of panic attack onset, it is difficult to dose a patient with an effective drug at a stage that is early enough to prevent or abort the panic attack. One way to rapidly deliver a drug into a patient's blood circulation is by intravenous injection. However, since panic attacks typically occur at unpredictable times, and because it is difficult for a patient to self-administer an IV injection, an IV method of administration is impractical to treat the onset of a panic attack. Conversely, drugs taken orally to prevent the onset of panic attacks would also not be practical, as orally administered drugs require too much time to enter systemic circulation, e.g., typically more than 30 minutes. As panic attack onset occurs typically in less than 10 minutes, oral drugs are also not ideal.

This being stated, the present invention is drawn to systems and methods of attenuating and/or reducing the occurrences of panic attacks. In accordance with this, a method for attenuating a panic attack can comprise steps of delivering a drug to a subject such that the drug forms a depot beneath a skin surface, wherein the drug is suitable for attenuating a panic attack if delivered in a pharmaceutically effective amount into systemic circulation of the subject. Once the depot is formed, the step of applying heat to the skin surface when the subject experiences an onset of the panic attack can be carried out, thereby causing the pharmaceutically effective additional dose of the drug to enter systemic circulation from the depot.

In another embodiment, a method for reducing the occurrence of panic attacks as well as attenuate panic attacks at their onset can comprise steps of delivering a drug to a subject having a history of panic attacks such that the drug generates a baseline level of the drug within systemic circulation of the subject, and forms a depot of the drug beneath a skin surface of the subject that is deliverable into systemic circulation upon application of heat. The drug can be suitable for reducing the occurrences of panic attacks when delivered at the baseline level, and can also be suitable for attenuating the symptoms of a panic attack upon onset when an extra dose of the drug is delivered from the depot into systemic circulation. A step of applying the heat to the skin surface when the symptoms of the panic attack are experienced can be carried out by the subject to treat the onset of the panic attack.

In another embodiment, a system for attenuating a panic attack can comprise a drug delivery device and a heating device. The drug delivery device can be configured to form a depot of drug beneath a skin surface of the subject. The drug can be suitable for attenuating a panic attack if a pharmaceutically effective amount is delivered into systemic circulation of the subject shortly after the onset of an attack is noticed, i.e. within 5 to 10 minutes. The heating device can be configured to apply heat, such as controlled heat, to the skin surface upon the onset of the panic attack, thereby causing the pharmaceutically effective amount of the drug to enter systemic circulation from the depot.

In still another embodiment, a system for reducing the occurrence of panic attacks as well as attenuate panic attacks at their onset can comprise a drug delivery device and a heating device. The drug delivery device can be configured to generate a baseline level of the drug within systemic circulation of a subject, and form a depot of the drug beneath a skin surface of the subject that is deliverable into systemic circulation upon application of heat. The drug can be suitable for reducing the occurrences of panic attacks when delivered at the baseline level, and can also be suitable for attenuating the symptoms of a panic attack upon onset when an additional dose is delivered from the depot into systemic circulation. The heating device can be configured to apply controlled heat to the skin surface upon the onset of the panic attack.

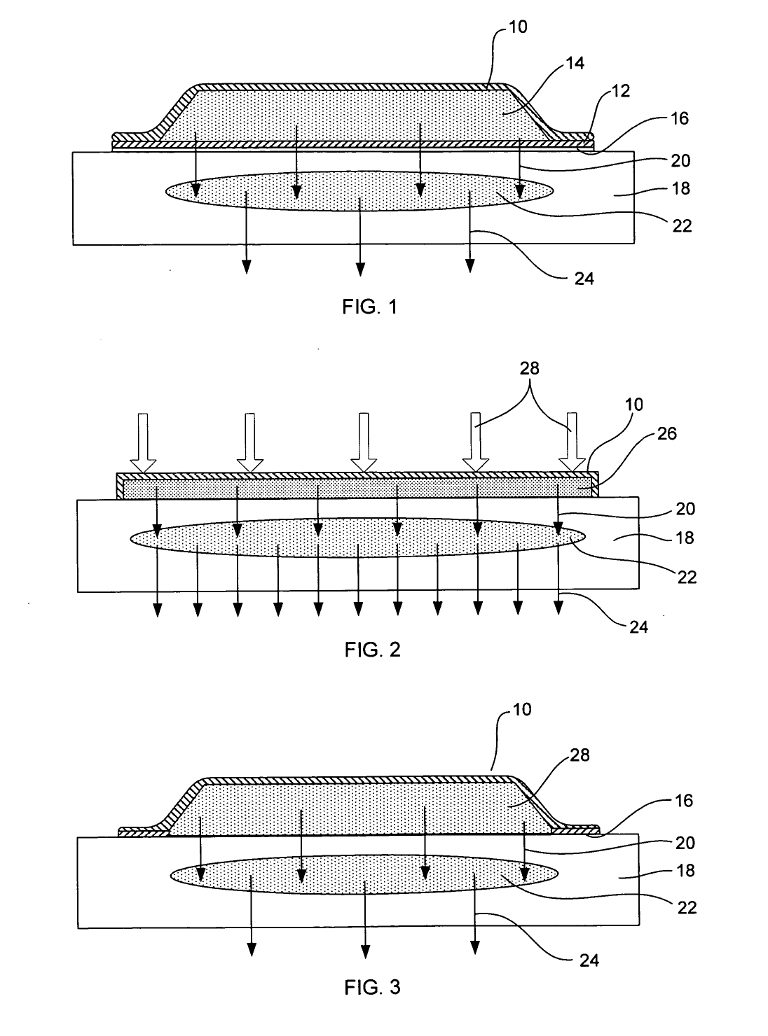

To exemplify certain systems in accordance with these embodiments, reference is now made to FIG. 1, which schematically depicts a reservoir transdermal delivery system having an impermeable backing 10, a rate-limiting membrane 12, a reservoir of a benzodiazepine drug solution 14, and a permeable adhesive layer 16 which can be incorporated as a continuous layer between the rate-limiting membrane and the skin, 18, or in a concentric or perimeter configuration around the membrane. The device is adhered to skin 18 and the benzodiazepine is transported through the rate limiting membrane and the adhesive, through the skin surface, and a portion then enters the systemic circulation via capillary blood vessels while another portion stays in the tissues close to the skin surface to form a drug depot 22. Though not shown, heat can be applied to the skin surface under which the depot exists to increase the levels of benzodiazepine delivered to systemic circulation, in accordance with embodiments of the present invention.

FIG. 2 depicts a similar system as described in FIG. 1, except that the drug delivery system is a matrix patch rather than a reservoir patch. Specifically, FIG. 2 schematically depicts a matrix transdermal delivery system having an impermeable backing 10 and a matrix 26 of a benzodiazepine drug solution and an adhesive. In this embodiment, the benzodiazepine-containing matrix is adhered directly to the skin 18 and the benzodiazepine is transported through the skin surface along a first delivery path 20, thereby forming a drug depot 22 beneath the skin surface, such as within the epidermis, dermis, and/or subcutaneous layer. A portion of the drug enters systemic circulation directly and a portion forms the depot, both of which permeate across the same portion of the skin. The increase in benzodiazepine delivery in the embodiment is provided by the application of heat 28 in accordance with embodiments of the present invention.

FIG. 3 depicts a similar system as described in FIG. 2, except that the drug delivery system is a gel patch rather than a matrix patch. Thus, the adhesion can be provided by perimeter adhesive rather than adhesive throughout the matrix. Additionally, the benzodiazepine drug is favorably positioned in the discontinuous oil phase of an emulsion or microemulsion, and a continuous aqueous is in the form of a semi-solid or gel. More specifically, FIG. 3 schematically depicts a gel patch transdermal delivery system having an impermeable backing 10, a gel of a benzodiazepine drug microemulsion or emulsion 28, and an adhesive layer 16 which is positioned in a concentric or perimeter configuration around gel at the contact point a between the skin and the impermeable backing. The device is adhered to skin 18 and the benzodiazepine is transported through the skin surface, wherein a portion enters the systemic circulation via capillary blood vessels while another portion stays in the tissues close to the skin surface to form a drug depot 22. Though not shown, heat can be applied to the skin surface under which the depot exists to increase the levels of benzodiazepine delivered to systemic circulation, in accordance with embodiments of the present invention.

With respect to the system and method for reducing the occurrences of panic attacks as well as attenuate panic attacks at their onset, while a baseline level of drug in the systemic circulation lowers the incidence of panic attacks, such levels will likely not prevent all episodes of panic attacks. Thus, the presence of a drug or drug depot beneath a skin surface can remain at the ready to be placed in systemic circulation almost immediately.

To illustrate one embodiment, one can consider a transdermal patch that delivers a depot of drug beneath the skin. When a patient wearing a transdermal patch feels the onset of a panic attack, the patient can place a controlled heating device on the skin above the depot, e.g., on or over the drug patch, to heat the skin. The increase in skin temperature can cause a dramatic increase in blood circulation in and around the depot. The increased circulation can then cause the drug in the depot to be rapidly “dumped” into the systemic circulation, resulting in a rapid increased drug concentration in the blood stream. The increased drug concentration in the blood stream can then cause a rapid increase in drug concentration at a site of action in the brain, which can attenuate or abort the panic attack. The magnitude and speed of the increase in drug concentrations in blood circulation by depot dumping can be effective in attenuating the panic attack at or close to its onset.

In another embodiment, a drug depot can be provided by methods other than by transdermal delivery. For example, a depot can be formed by injection or implantation of a drug beneath a skin surface. In one embodiment, an implant can be formed that includes a controlled or extended release mechanism. In one embodiment, the implant can form a depot that delivers drug systemically upon application of heat. In another embodiment, drug can be delivered in baseline levels from the depot, and the levels can be increased rapidly by the application of heat. Extended release of baseline levels of drug can be delivered for days, weeks, or even months. When a subject or patient feels the onset of a panic attack, the subject can apply, or have applied, a controlled heating device onto the skin area surrounding or in contact with the depot. As described, the heat can cause a greatly increased rate of release of the drug from the formulation into systemic circulation by increasing body fluid circulation surrounding the formulation and/or by changing the properties of the formulation if the release mechanism of the formulation is designed to be temperature sensitive, e.g., a heat sensitive thermogel.

Controlled Heating

A controlled heating device that can be used in accordance with systems and methods of the present invention can be configured to generate heat promptly when activated. Rapid heat generation can facilitate increasing drug delivery into systemic circulation in a quick enough manner to have an affect on a panic attack at an early stage. In one embodiment, the heating device can provide heat at a temperature greater than body temperature, but less than a temperature that would cause irreversible skin damage, i.e. burn the skin. An exemplary temperature range that can be implemented for use is from about 37° C. to about 47° C. Temperatures too much higher than about 47° C. can damage the skin, while temperatures much lower than 37° C. may not cause sufficient therapeutic effect. In one embodiment, a more preferred temperature range can be from about 40° C. to 43° C.

Application duration of heat can be determined by a specific need. Heating for an undesirably prolonged period may not lead to added efficacy due to rapid delivery, or may waste the drug and cause overdose-related adverse side effects. On the other hand, too short of a heating duration may not lead to adequate release of the drug from the depot into systemic circulation, and thus, may not be effective in treating the panic attack. In one embodiment, the application time can be from 1 to 30 minutes, and in a more specific embodiment, from 5 to 15 minutes. In either embodiment, there are advantages to application of the heat within 5 or 10 minutes of the onset of the panic attack.

A controlled heating device for use in accordance with embodiments of the present invention can generate and provide heat by one of a number of mechanisms. One mechanism involves generating heat by oxidation of certain metals, such as iron. Such a mechanism can be configured to generate heat by an oxidation reaction between a component, e.g., iron, within the controlled heating device and oxygen in ambient air. A heating device in accordance with this embodiment is described more fully in Examples 9 and 10 hereinafter. U.S. patent application Publication No. 09/878,558, which is incorporated herein by reference in its entirety, also describes such heating devices. Other heating mechanisms can also be used, such as electric heating, heating by phase transition (such as phase transition of sodium acetate solutions), infrared heating, and microwave heating.

Active Ingredients and Formulations for Treating Panic Disorder

Another aspect of the current invention relates to active ingredients for transdermal delivery of benzodiazepines and other drugs or active ingredients that can attenuate panic attacks at their onset, and/or reduce the number of panic attacks from occurring. Benzodiazepines are a class of drugs that are widely used to treat panic attacks but have generally been orally administered have not been particularly successful in attenuating the onset of a panic attack. However, if one or more of such drugs could be delivered rapidly into the systemic circulation at the onset of a panic attack, the onset of a panic attack could be attenuated or aborted. Typical benzodiazepines used for these purposes include alprazolam, lorazepam, clonazepam, diazepam, and midazolam, although other benzodiazepines may also be effective. As stated, these drugs can be used to form a depot beneath a skin surface, such as by transdermal delivery, injection, or implantation.

As described “flux” can be defined as the quantity of drug that permeates across the skin per unit area per unit time. To illustrate, polyisobutylene (PIB) glue is a common component used in patches for transdermal drug delivery, and PIB glue based patches produce satisfactory transdermal fluxes for many other drugs. However, alprazolam in PIB formulation generated extremely low transdermal flux. One reason for such poor transdermal fluxes can be due to low solubility. Alprazolam has extremely low solubility in water and some solvent based pressure-sensitive adhesives, which at least partially explains why water-based and PIB-based transdermal alprazolam formulations also produce such low fluxes.

Through experimentation, it has been found that alprazolam has at least 5 times higher solubility than water in the following liquids: eugenol (clove oil), rose oil, n-methyl-pyrrolidone, isopropyl myristate, ethanol, oleyl alcohol, citronella oil, and isopropyl alcohol, Labrasol, wintergreen oil, octyldodecanol, ethyl oleate, evening primrose oil, and orange oil. Further, the following liquids provided at least 20 times higher solubility than water: wintergreen oil, octyldodecanol, oleyl alcohol, ethanol, citronella oil, rose oil, eugenol, n-methyl pyrrolidone, isopropyl alcohol. Still further, the following liquids provided greater than 100 times higher solubility than water: ethanol, citronella oil, rose oil, n-methyl pyrrolidone, and isopropyl alcohol. It is to be emphasized that the above list of solubilizing agents is specific to alprazolam. As such, this list is applicable to this particular drug. This being stated, still, some of the solubilizing agents listed as having favorable solubilizing properties may work well with other lipophilic drugs, in accordance with embodiments of the present invention, as would be easily ascertainable to one skilled in the art. Though it is useful to know what compositions can be used to solubilize these and other similarly soluble medications for use in dermal delivery devices, applying a liquid formulation directly on the skin can be impractical in some devices, such as dermal delivery devices. This is because variable drug delivery quantities typically occur which can be caused by poorly defined contact area with the skin. Further, liquid formulations are vulnerable to be wiped from the skin by external objects, such as clothing.

One solution to this problem is solubilize the active ingredient or drug in one of the solubilizing liquids described above, and then including the solubilized drug in the form of a gel for transdermal delivery. However, the liquids listed as being capable of solubilizing alprazolam are not known to be substances that can be gelled. Another approach would be to incorporate a liquid formulation, including the active ingredient solubilized in the solvent, into a reservoir patch configuration. In a typical reservoir patch, the drug formulation is held in a thin compartment with one side of the compartment including a rate limiting membrane which is permeable to the drug. The rate limiting membrane is typically adhered to the skin by an adhesive that will allow the drug to pass and contact the skin. In other words, the drug can permeate the rate limiting membrane and the adhesive layer before it reaches the skin. However, it has been found that certain solubilizing liquids in an alprazolam formulation can significantly interact with the adhesive layer, rendering this approach difficult for practical application. Moreover, the incompatibility between the formulation component, i.e. solubilizing liquids, and the adhesive layer can also be an issue with other formulations. In general, in order to be effective, the adhesive layer of a reservoir patch should be permeable to the drug in the formulation and be compatible with the ingredients of the formulation over the shelf-life of the product.

Formulations that have been discovered to be effective for providing dosing of a benzodiazepine by transdermal delivery include the use of emulsions. In a first embodiment, a liquid source that can solubilize the benzodiazepine can be selected for use. The liquid source is generally an oil that is immiscible in water. The benzodiazepine can be at least partially dissolved in the oil to form an oil phase. A water-based solution can also be prepared that includes at least one gelling agent that can be used to form a gel of the aqueous phase. The oil phase and the aqueous phase can then be emulsified. Appropriate emulsifying agent(s) can be used if desired. Once in an emulsified stage, the aqueous phase can then be gelled using a composition interactive with the gelling agent.

To illustrate a specific embodiment of this formulation, the following preparative scheme can be carried out. An aqueous phase can be prepared as follows, (a) dissolve 20 wt % polyvinyl alcohol (PVA, gelling agent) in water and (b) dissolve 0.4% Pemulen TR2 (Acrylates/C10-30 alkyl acrylate crosspolymer, emulsifying agent, from Noveon, Inc., Cleveland, Ohio) in water. Mix the two aqueous solutions in a 1:1 ratio until thoroughly mixed. An oil phase can be prepared by dissolving an excess amount of alprazolam into euqenol. The oil phase with the drug present can then be added to the aqueous phase and agitated to form an emulsion. Though the emulsifying agent is present in the aqueous phase, it can likewise or alternatively be included in the oil phase. Alternatively, the emulsifying agent can be admixed therein when the oil phase and the aqueous phase are combined. The emulsion, once formed, can then be cast onto a fabric material impregnated with sodium borate, which permeates into the cast emulsion layer and acts to gel the aqueous phase by causing a crosslinking reaction with the polyvinyl alcohol. Since the aqueous phase is the continuous phase and the oil phase is the discontinuous phase in the emulsion formulation, the gelling of the aqueous phase solidifies the entire formulation into a soft solid and coherent layer. In this state, the composition can be applied to the skin for delivery of the benzodiazepine active agent.

In accordance with this manufacturing method, in one embodiment, the emulsion can be gelled by a crosslinking process within 30 minutes. Once the oil phase and the aqueous phase are emulsified, and once the aqueous phase is gelled, the emulsion will remain as formed due to the solid characteristics of the gelled aqueous phase. As such, the use of an emulsifying agent is not strictly required, provided the aqueous phase can be sufficiently gelled before phase separation can occur. The slowing of phase separation may be helped by increasing the viscosity of the aqueous phase by adding viscosity increasing agents. By removing the requirement of the use of an emulsifying agent, more freedom in selecting other ingredients of the formulations can be realized. Further, though gelling is described as one means of solidifying the emulsion, other techniques can be used, including the use of freeze-thaw cycles, e.g., using polyvinyl alcohol, or radiation, e.g., using polyvinyl pyrrolidone, acrylamide 2-methyl 1-propane sulfonic acid, and hydroxyl cyclohexyl phenyl ketone.

The aqueous phase of the oil-in-water emulsion can also be gelled by using a gelling agent in the aqueous phase to form a thermo-reversible gel. Such a gel can be configured to liquefy when heated and re-solidify after cooling. For example, using carrageenin as a gelling agent in water can produce a gel that melts when heated, i.e. above 60° C., and solidify when it is cooled. A heated or melted form of such an emulsion formulation can be fluid and can be cast into a thin layer. Cooling of such a layer can solidify the formulation.

Agents for increasing the tackiness of the gelled formulation may be also added. These agents include, but not limited to, polyvinyl pyrrolidone, acrylic polymers, and their derivatives.

Another type of practical system for transdermal delivery of benzodiazepines involves incorporating the drug into a microemulsion system, and placing the microemulsion system into a reservoir patch configuration. Microemulsions are defined as a system of water, oil, and surfactants which, when mixed, form a single transparent isotropic and thermodynamic stable liquid solution. Depending on the physico-chemical properties of the components, formation of microemulsions can appear over a wide range of oil-water-surfactant compositions.

Conditions that are typically met in order to obtain a microemulsion include production of a low oil-water interfacial tension; formation of a highly fluid interfacial surfactant film; and association of oil phase molecules with interfacial surfactant film. The main advantage of the use of microemulsion formulations is the ability to increase the activity of the drug by incorporating it into the water or oil phase (depending on the lipophilicity of the drug).

A consideration that can be used in preparing a microemulsion formulation includes determining the solubility of benzodiazepine in various liquids, and selecting an appropriate liquid(s) for use. Liquids of acceptable solubility are identified as potential components for inclusion in the microemulsion formulation. In one embodiment, the oil and/or surfactant components chosen can then mixed together in several different fixed ratios. Water (with or without a fixed amount of co-surfactant) can then be added and gently agitated to the oil/surfactant mixture until the microemulsion phase is formed. As relatively large amounts of surfactants are typically used in microemulsion systems, and as surfactants can interfere with the function of certain gelling agents, microemulsion formulations typically are not as easily gelled into a matrix patch. This being stated, microemulsions are not precluded from use in gel patches as well as reservoir patches.

### EXAMPLES

The following example illustrates the embodiments of the invention that are presently best known. However, it is to be understood that the following is only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following example provides further detail in connection with what is presently deemed to be the most practical and preferred embodiments of the invention.

### Example 1

### Skin Permeation Methodology

In order to assess the influence of solvents on skin permeability of alprazolam, in vitro skin flux of alprazolam in various liquid formulations was tested. All liquid formulations contained excess alprazoiam in solution. In the study, hairless mouse skin (HMS) was used for the in vitro testing. Freshly separated epidermis removed from the abdomen was mounted carefully between two cells of a Franz diffusion cell. The receiver chamber of the cell was filled with pH 7.4 phosphate buffered saline (PBS). The experiment was initiated by placing the test formulation on the stratum corneum (SC) side of the HMS. Franz cells were placed in a Franz diffusion cell console (Logan Instruments Corp. Model #: FDC-24) maintained at 37° C. At predetermined time intervals, an 800 μL aliquot was withdrawn and replaced with fresh PBS solution. Skin flux (μg/cm²/h) was determined from the steady-state slope of a plot of the cumulative amount of benzodiazepine that permeates versus time.

The steady-state flux of alprazolam from the test formulations through HMS maintained at 37° C. is presented in Table 1 below.

TABLE 1

| Formulation | | Skin Flux |
|---|---|---|
| Number | Formulation | (μg/cm²/h) |
| 1 | PBS alone | 0.1 ± 0.07 |
| 2 | 20% ethanol | 1.0 ± 0.6 |
| | 80% PBS | |
| 3 | 40% ethanol | 4 ± 1 |
| | 60% PBS | |
| 4 | 25% IPM | 3 ± 1 |
| | 30% Ethanol:water (1:1) | |
| | 45% Tween 80 | |
| | (microemulsion) | |
| 5 | 100% Eugenol | 4 ± 1 |
| 6 | 100% poly ethylene glycol | 0.2 ± 0.08 |
| | 400 (PEG 400) | |

As can be seen in Table 1, simply putting alprazolam in an aqueous solution (PBS) or in PEG 400 resulted in formulations that produced far from sufficient flux, assuming 0.5 mg to 7 mg alprazolam per day can be used to effectively prevent panic disorder. The use of ethanol, which has excellent solvent properties for alprazolam, significantly increases the flux. The microemulsion formulation also produced adequate flux.

### Example 2

### In Vitro Skin Flux of Alprazolam from PVA Hydrogels

Several polyvinyl alcohol hydrogel formulations with excess alprazolam were prepared as follows:

Formulation 1

Part A: 5 wt % eugenol in water emulsion, 0.4 wt % TR-2 emulsifier, and excess amount of alprazolam.

Part B: 17 wt % polyvinyl alcohol in water.

Formulation 1 was obtained by aggressively mixing one weight portion of Part A with one weight portion of Part B.

Formulation 2

Part A: 10 wt % eugenol in water emulsion, 0.4 wt % TR-2 emulsifier, and excess amount of alprazolam.

Part B: 17 wt % polyvinyl alcohol in water.

Formulation 2 was obtained by aggressively mixing one weight portion of Part A with one weight portion of Part B.

Formulation 3

Part A: emulation of 34 wt % IPM (isopropyl myristate), 24 wt % ethanol, 24 wt % water, 18 wt % Tween 80, and excess amount of alprazolam.

Part B: 17 wt % polyvinyl alcohol in water.

Formulation 3 was obtained by aggressively mixing one weight portion of Part A with one weight portion of Part B.

Formulation 4

6 wt % PVA, 34 wt % water, 60 wt % N-methyl pyrrolidone (NMP), and excess amount of alprazolam.

Formulation 5

13.5 wt % polyvinyl alcohol, 77.5 wt % water, 10 wt % N-methyl pyrrolidone (NMP), and excess amount of alprazolam.

Formulation 6

28% polyvinyl alcohol, 65% water, 7% rose oil, and excess alprazolam.

Each of the six viscous solutions were disposed on to 25 cm² piece of Dexter nonwoven material that was pretreated with 1 mL of a 2 wt % sodium borate solution. In each case, the solutions formed a solidified gel within approximately 30 minutes. Each of the gelled formulations was cut into a 2 cm² piece and placed on a stratum corneum (SC) and mounted on a diffusion cell for flux measurements, as described in Example 1. The results of these tests are presented in Table 2 below.

TABLE 2

| | Ingredients in addition | |
|---|---|---|
| | to excess amount of | Skin Flux |
| Formulation | alprazolam | (μg/cm²/h) |
| 1 | 2.5% Eugenol | 1.3 ± 1.1 |
| | 0.2% TR-2 | |
| | 8.5% PVA in water | |
| 2 | 5% Eugenol | 5 ± 1 |
| | 0.2% TR-2 | |
| | 8.5% PVA in water | |
| 3 | 17% IPM | 0.5 ± 0.2 |
| | 12% ethanol | |
| | 9% Tween 80 | |
| | 8.5% PVA | |
| | 53.5% water | |
| 4 | 6% PVA | 1.1 ± 0.1 |
| | 60% NMP | |
| | 34% water | |
| 5 | 13.5% PVA | 0.3 ± 0.07 |
| | 10% NMP | |
| | 76.5% water | |
| 6 | 28% PVA | 3 ± 1 |
| | 7% Rose Oil | |
| | 65% water | |

As can be seen in Table 2, even relatively small amounts of eugenol or rose oil in the formulation produced a significantly increased flux. The formulations described in the present example can be gelled into a thin layer for incorporation into a matrix patch or a gel patch.

### Example 3

### In Vitro Skin Flux of Alprazolam from Laminated Composites

Varying amounts of alprazolam and a solvent-based polyisobutylene adhesive (Adhesives Research MA-31) were mixed thoroughly to obtain a homogenous suspension. A 50 micron thick film of this mix was cast on a release liner (3M 1022) with an eight path applicator. The cast film was left overnight to allow the organic solvents (toluene, vinyl acetate, ethyl acetate, and ethanol) to evaporate. The following day, a backing film (3M CoTran 9720) was placed over the formulation, and the film was die cut into 2 cm² section. The release liner was removed, and the film was placed on an excised stratum corneum (SC) layer of the HMS and mounted on a diffusion cell for flux measurements, as described in Example 1. The results of these tests are presented in Table 3 below.

TABLE 3

| | Skin Flux |
|---|---|
| Formulation | (μg/cm²/h) |
| 0.5% alprazolam + MA-31 | 0.4 ± 0.1 |
| 3.5% alprazolam + MA-31 | 0.5 ± 0.07 |

As can be seen from this example, the addition of alprazolam into a PIB adhesive yields inadequate flux.

### Example 4

### In Vitro Skin Flux of Lorazepam Formulations from Various Liquid Formulations

Lorazepam formulations were prepared and tested as presented in Example 1. The details of these formulations and the results of the in vitro flux experiments are shown below in Table 4.

TABLE 4

| | Skin Flux |
|---|---|
| Formulation | (μg/cm²/h) |
| PBS alone | 0.1 ± 0.07 |
| 30% ethanol | 1.4 ± 0.7 |
| 70% PBS | |
| 60% ethanol | 5 ± 1 |
| 40% PBS | |
| 25% IPM | 8 ± 2 |
| 30% EtOH:water (1:1) | |
| 45% Tween 80 | |
| (microemulsion) | |
| 27% NMP - 73% PEG 400 | 2.6 ± 0.5 |

The estimated daily dose of lorazepam is 0.5 to 4 mg/day. As can be seen from Table 4, an aqueous based formulation produces insufficient flux. Addition of ethanol to solubilize lorazepam significantly increases skin flux. The microemulsion also produces adequate flux. Further, the addition of NMP, another acceptable solubilizer of lorazepam, into PEG-400 also produces adequate skin flux.

### Example 5

### In Vitro Skin Flux of Lorazepam from Polyvinyl Alcohol Hydrogels

A lorazepam polyvinyl alcohol formulation was prepared by adding 5 wt % lorazepam to a 15 wt % polyvinyl alcohol in water solution, and the mixture was agitated. The solution was capped and left at room temperature for 24 hours. After 24 hours, the sample was gelled and the in vitro flux was determined as outlined in Example 2. The details of these formulations and the results of the in vitro flux experiments are shown below in Table 5.

TABLE 5

| | Skin Flux |
|---|---|
| Formulation | (μg/cm²/h) |
| 5% Lorazepam | 0.21 ± 0.04 |
| 15% PVA | |

The result depicted in Table 5 suggests that lorazepam does not have sufficient activity (solubility) in the PVA gel to provide sufficient flux. The in vitro flux of lorazepam formed a 15 wt % polyvinyl alcohol solution, which is not different from the lorazepam in vitro flux from PBS in Table 4.

### Example 6

### In Vitro Skin Flux of Lorazepam from Laminated Composites

Lorazepam dispersed in silicone PSA was prepared as outlined in Example 3. BIO-PSA 74301 (Dow Corning) is a silicone based pressure sensitive adhesive with heptane as the evaporative solvent. The details of these formulations and the results of the in vitro flux experiments are shown below in Table 6.

TABLE 6

| | Skin Flux |
|---|---|
| Formulation | (μg/cm²/h) |
| 5% Lorazepam in BIO-PSA | 0.12 ± 0.03 |
| 7-4301 | |

The results indicate that silicone based PSA does not produce sufficient lorazepam flux.

### Example 7

### In Vitro Skin Flux of Clonazepam Formulations from Liquid Formulations

Clonazepam formulations were prepared and tested as in Example 1. The details of these formulations and the results of the in vitro flux experiments are shown below in Table 7.

TABLE 7

| | Skin Flux |
|---|---|
| Formulation | (μg/cm²/h) |
| PBS alone | 0.05 ± 0.004 |
| 95% PBS | 0.05 ± 0.001 |
| 5% HPβCD | |
| 99.8% PBS | 0.06 ± 0.007 |
| 0.2% PVP (K-90) | |
| 25% IPM | 1.5 ± 0.4 |
| 30% EtOH:water (1:1) | |
| 45% Tween 80 | |
| (microemulsion) | |
| 27% NMP | 1.0 ± 0.4 |
| 0.1% Oleic Acid | |
| 72.9% PEG 400 | |
| 40% NMP | 0.2 ± 0.1 |
| 60% PBS | |

The daily dose of clonazepam can be from 0.5 to 4 mg per day. Clonazepam flux from an aqueous based formulations (Formulation Numbers 1-3, and 6) containing the solubilizing agents HPβCD and NMP, as well as a super saturation agent (PVP) do not possess sufficient flux. The PEG based formulation (Formulation Number 5) and the microemulsion (Formulation Number 4) produced sufficient flux.

### Example 8

### In Vitro Skin Flux of Clonazepam Formulations from Laminated Composites

Clonazepam dispersed in silicone PSA was prepared as outlined in Example 3. BIO-PSA 7-4301 (Dow Corning) is a silicone based pressure sensitive adhesive with heptane as the evaporative solvent. Clonazepam formulations were prepared and tested as in Example 3. The details of these formulations and the results of the in vitro flux experiments are shown below in Table 8.

TABLE 8

| | Skin Flux |
|---|---|
| Formulation | (μg/cm²/h) |
| 1.7% Clonazepam in BIO- | No flux |
| PSA 7-4301 | |
| 1.7% Clonazepam | No flux |
| 0.3% Oleic Acid in BIO-PSA | |
| 7-4301 | |
| 10% Clonazepam | 0.37 ± 0.09 |
| 4% NMP in BIO-PSA 7-4301 | |

None of the formulations listed in Table 8 had sufficient clonazepam flux. These drug-in-adhesive formulations using clonazepam did not include an appropriate solubilizer, e.g., NMP, in order to achieve adequate flux.

### Example 9

### Typical Iron-Oxidation Based Controlled Heating Device (CHADD)

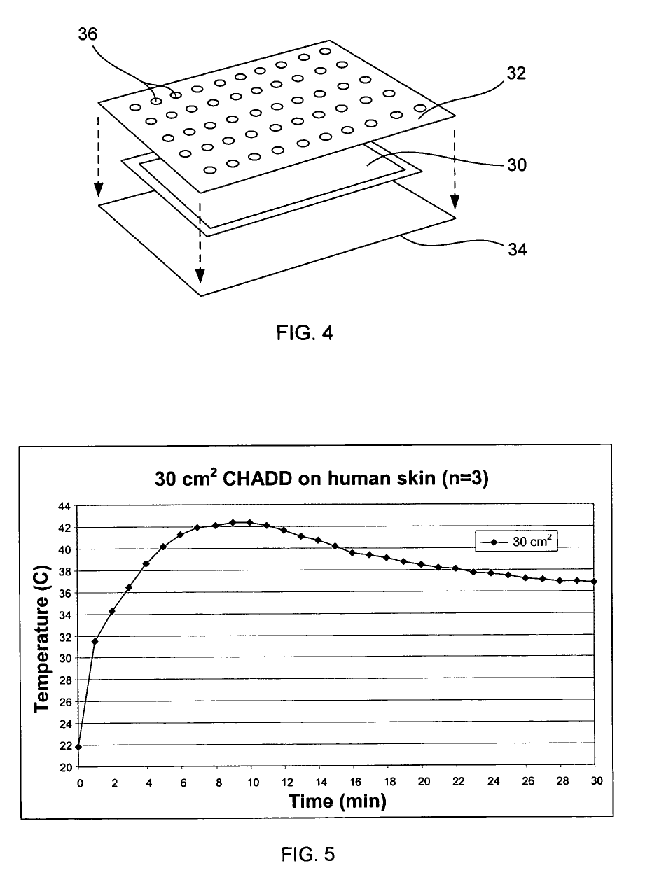

A CHADD controlled heating device, shown generally in FIG. 4, was prepared, which is a thin, flexible, patch that can provide controlled heat to elevate skin temperature to a range from about 37° C. to 47° C. for approximately 15 minutes. The CHADD heating patch contained a heating pod 30 having a heat-generating medium. In this case, the heat-generating medium included a powder-filled pouch composition capable of an exothermic iron oxidation reaction. The heating pod was laminated between two air impermeable films, including a top cover film 32 and a bottom cover film 34, which were ultimately sealed (not shown) at the edges. The top cover had a finite number of precisely sized holes 36 (not to scale) to control the amount of ambient oxygen that can enter into the heating pod. In this embodiment, the CHADD heating patch was designed to have a heating area of 30 cm². The top cover film and the bottom cover film can each be prepared from single-coated medical tape, for example. The top cover film can function to regulate the heat generation, and the bottom cover film can be impermeable to oxygen, i.e. no holes. The heating pod can be prepared from heat-sealable filter paper filled with the heat-generating medium. When the CHADD heating patch was removed from the airtight pouch, oxygen in the ambient air flowed into the heat generating medium via the holes on the top cover and started an exothermic iron oxidation reaction. The reaction rate, which controlled the heating temperature profile, was determined by the number, size, and configuration of the holes. After an initial rise in temperature, the temperature of the skin and sub-skin tissues reached and remained within a controlled temperature range for a pre-determined period of time. When the heat generating medium became exhausted, the temperature gradually returned to normal.

An exemplary composition that can be used to generate heat upon interaction with ambient oxygen is set forth in Table 9 as follows:

TABLE 9

| Ingredient | Weight % | Function |
|---|---|---|
| Iron powder | 12.7 wt % | reducing agent |
| Activated carbon | 31.9 wt % | water retaining agent |
| Wood flour | 19 wt % | bulking agent |
| Sodium chloride | 12.7 wt % | reaction catalyst |
| Deionized water | 23.7 wt % | reaction catalyst |

The CHADD heating pod 30 was formed by putting 4 grams of the mixed powder described above into a filter paper pouch. The CHADD heating pod was then placed onto the bottom cover film 34 and water was dispensed onto the CHADD heating pod. The top cover film 32 was then immediately laminated onto the top of the CHADD heating pod. The finished CHADD heating patch was immediately packaged in a heat-sealed barrier film pouch. A temperature profile of a CHADD heating patch as tested on human skin is provided as FIG. 5.

### Example 10

### Use of a CHADD Heating Patch to Delivery a Benzodiazepine from a Transdermal Delivery Patch

A patient who suffers from a panic disorder wears a transdermal alprazolam patch that is configured to deliver baseline levels of alprazolam to decrease the incidence of panic attacks. The transdermal alprazolam patch delivers alprazolam into the systemic circulation and produces relatively constant blood levels of alprazolam to the patient. Though the baseline level of alprazolam decreases the incidence of panic attacks, in practice, the baseline levels do not completely prevent all occurrences of panic attacks. As the baseline levels are being delivered, a portion of the alprazolam becomes absorbed into the skin and becomes stored just beneath the skin surface, e.g., in the epidermis, dermis, and/or subcutaneous layer, and/or sub-skin tissues in the form of an alprazolam depot. As not all panic attacks are typically prevented by the administration of baseline alprazolam concentrations, when the patient feels the onset of the panic attack while wearing the patch, heat can be immediately applied to release alprazolam from the depot into systemic circulation, thereby rapidly increasing systemic levels of the drug. A CHADD heating device (similar to that described in Example 9) on top of the alprazolam patch can be used to provide the desired heat. The CHADD heating device, after being taken out of an air-tight container, generates heat via reaction of the iron power in the unit and ambient oxygen. The skin underneath the alprazolam patch can be heated to a temperature range from 39° C. to 43° C. for about 15 minutes. The increased skin temperature can dramatically increase the blood flow in the tissues in which the depot exists, and can rapidly carry the alprazolam from the depot into the blood circulation. As a result, the blood alprazolam concentration is significantly increased within a few minutes from the application of the CHADD heating patch, and the panic attack can be successfully attenuated or aborted.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A method for attenuating a panic attack, comprising steps of:
delivering a drug to a subject such that the drug forms a depot beneath a skin surface, said drug suitable for attenuating a panic attack if delivered in a pharmaceutically effective amount into systemic circulation of the subject; and
applying heat to said skin surface when said subject experiences an onset of the panic attack, thereby causing the pharmaceutically effective amount of the drug to enter systemic circulation from the depot.

2. The method of claim 1, wherein said subject has a history of at least one panic attack.

3. The method of claim 1, wherein said depot is located within 0.5 cm of said skin surface.

4. The method of claim 1, wherein the step of applying heat is within 5 minutes from the onset of the panic attack.

5. The method of claim 1, wherein the skin surface is heated at from 37° C. and 47° C.

6. The method of claim 1, wherein the step of delivering is by applying the drug to the skin surface, and wherein the drug transdermally forms the depot.

7. The method of claim 1, wherein the step of delivering is by implanting the drug below the skin surface to form the depot.

8. The method of claim 1, wherein the step of applying heat is achieved using a controlled heating device which generates heat by an oxidation reaction between a component within said controlled heating device and oxygen in ambient air.

9. The method of claim 8, wherein said component within said controlled heating device is iron.

10. The method of claim 1, wherein the step of applying heat is achieved using a controlled heating device which generates heat through electric heat, microwave heat, or exothermic phase transition.

11. The method of claim 1, wherein the drug is a benzodiazepine.

12. The method of claim 11, wherein the benzodiazepine is selected from the group consisting of alprazolam, lorazepam, clonazepam, midazolam, and combinations thereof.

13. A method for reducing the occurrence of panic attacks as well as attenuate panic attacks at their onset, comprising steps of:
delivering a drug to a subject having a history of panic attacks such that the drug:
i) generates a baseline level of the drug within systemic circulation of the subject, and
ii) forms a depot of the drug beneath a skin surface of the subject that is deliverable into systemic circulation upon application of heat,
said drug being suitable for reducing the occurrences of panic attacks when delivered at the baseline level, and said drug also being suitable for attenuating the symptoms of a panic attack upon onset when an extra dose of it is delivered from the depot into systemic circulation; and
applying the heat to said skin surface when said symptoms of the panic attack are experienced by the subject.

14. The method of claim 13, wherein said depot is located within 0.5 cm of said skin surface.

15. The method of claim 13, wherein the step of applying heat is within 5 minutes from the onset of the panic attack.

16. The method of claim 13, wherein the skin surface is heated at from 37° C. and 47° C.

17. The method of claim 13, wherein the step of delivering is by applying the drug to the skin surface, and wherein the drug transdermally forms the depot.

18. The method of claim 13, wherein the step of delivering is by implanting the drug below the skin surface to form the depot.

19. The method of claim 13, wherein the step of applying heat is achieved using a controlled heating device which generates heat by an oxidation reaction between a component within said controlled heating device and oxygen in ambient air.

20. The method of claim 19, wherein said component within said controlled heating device is iron.

21. The method of claim 13, wherein the step of applying heat is achieved using a controlled heating device which generates heat through electric heat, microwave heat, or exothermic phase transition.

22. The method of claim 13, wherein the drug is a benzodiazepine.

23. The method of claim 22, wherein the benzodiazepine is selected from the group consisting of alprazolam, lorazepam, clonazepam, midazolam, and combinations thereof.

24. A system for attenuating a panic attack, comprising:
a drug delivery device configured to form a depot of drug beneath a skin surface of a subject, said drug suitable for attenuating a panic attack if delivered in a pharmaceutically effective amount into systemic circulation of the subject; and
a heating device configured to apply heat to the skin surface upon the onset of the panic attack, thereby causing the pharmaceutically effective amount of the drug to enter systemic circulation from the depot.

25. The system of claim 24, wherein said system is configured for delivery to the subject when the subject has a history of at least one panic attack.

26. The system of claim 24, wherein said depot is located within 0.5 cm of said skin surface.

27. The system of claim 24, wherein the step of applying heat is within 5 minutes from the onset of the panic attack.

28. The system of claim 24, wherein the skin surface is heated at from 37° C. and 47° C.

29. The system of claim 24, wherein the step of delivering is by applying the drug to the skin surface, and wherein the drug transdermally forms the depot.

30. The system of claim 24, wherein the step of delivering is by implanting the drug below the skin surface to form the depot.

31. The system of claim 24, wherein the step of applying heat is achieved using a controlled heating device which generates heat by an oxidation reaction between a component within said controlled heating device and oxygen in ambient air.

32. The system of claim 31, wherein said component within said controlled heating device is iron.

33. The system of claim 24, wherein the step of applying heat is achieved using a controlled heating device which generates heat through electric heat, microwave heat, or exothermic phase transition.

34. The system of claim 24, wherein the drug is a benzodiazepine.

35. The system of claim 34, wherein the benzodiazepine is selected from the group consisting of alprazolam, lorazepam, clonazepam, midazolam, and combinations thereof.

36. A system for reducing the occurrence of panic attacks as well as attenuate panic attacks at their onset, comprising:
a drug delivery device configured to:
i) generate a baseline level of the drug within systemic circulation of a subject, and
ii) form a depot of the drug beneath a skin surface of the subject that is deliverable into systemic circulation upon application of heat,
said drug suitable for reducing the occurrences of panic attacks when delivered at the baseline level, and said drug also suitable for attenuating the symptoms of a panic attack upon onset when an extra dose of it is delivered from the depot into systemic circulation; and
a heating device configured to apply the heat to the skin surface upon the onset of the panic attack.

37. The system of claim 36, wherein said subject has a history of at least one panic attack.

38. The system of claim 36, wherein said depot is located within 0.5 cm of said skin surface.

39. The system of claim 36, wherein the step of applying heat is within 5 minutes from the onset of the panic attack.

40. The system of claim 36, wherein the skin surface is heated at from 37° C. and 47° C.

41. The system of claim 36, wherein the step of delivering is by applying the drug to the skin surface, and wherein the drug transdermally forms the depot.

42. The system of claim 36, wherein the step of delivering is by implanting the drug below the skin surface to form the depot.

43. The system of claim 36, wherein the step of applying heat is achieved using a controlled heating device which generates heat by an oxidation reaction between a component within said controlled heating device and oxygen in ambient air.

44. The system of claim 43, wherein said component within said controlled heating device is iron.

45. The system of claim 36, wherein the step of applying heat is achieved using a controlled heating device which generates heat through electric heat, microwave heat, or exothermic phase transition.

46. The system of claim 36, wherein the drug is a benzodiazepine.

47. The system of claim 46, wherein the benzodiazepine is selected from the group consisting of alprazolam, lorazepam, clonazepam, midazolam, and combinations thereof.
